(19) United States
(12) Patent Application Publication
(10) Pub. No.: US 2024/0404708 A1
(43) Pub. Date: Dec. 5, 2024

(54) ALERT METHOD AND SYSTEMS ANALYZING HOUSEHOLD BEHAVIOR

(71) Applicant: Shimon BEN-DAVID, Binyamina (IL)

(72) Inventor: Shimon BEN-DAVID, Binyamina (IL)

(21) Appl. No.: 18/695,469

(22) PCT Filed: May 19, 2022
(86) PCT No.: PCT/IL2022/050527

§ 371 Date: Mar. 26, 2024

Related U.S. Application Data
(60) Provisional application No. 63/286,187, filed on Dec. 6, 2021.

(51) Int. Cl. G16H 50/30; G16H 40/67; G16H 50/20; G16H 50/70; H04B 17/318

(52) U.S. Cl. CPC G16H 50/30; G16H 40/67; G16H 50/20; G16H 50/70; H04B 17/318

(57) ABSTRACT

The present invention discloses a system for monitoring and alert, implemented by plurality of modules comprising one or more processors, operatively coupled to non-transitory computer readable storage devices, comprising the steps of: —analysis module configured for aggregating and/or synchronizing, filtering and calculating intensity changes and or creating data patterns template for each data type from data meters of electricity, waterflow gas data and all types of data communication flow: —data pattern analyse module, configured to identify anomaly between the current measured pattern data and compare it with the template saved in the memory, on the same time and day of the week, two weeks or three weeks. —alert module configured for determining alerts based on identifying anomaly in data patterns.

### TECHNICAL FIELD

The present invention relates to the field of analyzing alerting anomality human behavior-based energy measurements in households, more particularly the anaclasis is based water, flow, gas flow, RSSI (Received Signal Strength Indicator) and data flow.

### BACKGROUND ART

In recent years more and more nursing homes are closing and undergoing personal care at the elderly home. Supervision is required in order to provide a solution for cases which the patient undergoes any events that requires medical treatment. Mandatory requirement is that, the supervision application should not infringe the privacy of these people.

There are a variety of solutions that combine voice and video systems that include face and movement recognition etc., these systems infringe the privacy of the users and cannot cover the entire rooms at home.

In addition, there are distress buttons which are located at specific place at home and even on the user's neck. In many cases the user is not by the distress button or is unaware of his condition, and sometimes the event is unexpected so this solution is limited and does not provide a complete solution.

### SUMMARY OF INVENTION

The present invention discloses a system for monitoring and alert, implemented by plurality of modules comprising one or more processors, operatively coupled to non-transitory computer readable storage devices, comprising the steps of:
analysis module configured for aggregating and/or synchronizing, filtering and calculating intensity changes and or creating data patterns template for each data type from data meters of electricity, waterflow gas data and all types of data communication flow;
data pattern analyse module, configured to identify anomaly between the current measured pattern data and compare it with the template saved in the memory, on the same time and day of the week, two weeks or three weeks.
alert module configured for determining alerts based on identifying anomaly in data patterns.

According to some embodiments of the present invention the analysis module further comprising the step of analyzing user movement based on all types of wireless devices (modems) RSSI measurements.

According to some embodiments of the present invention the analysis module further comprising the step of identifying real time human activity and their association with the hours of the day by identifying specific events that reflect human activities of opening or closing or changing setting of electrical appliance, gas and water for differentiating between repeating (cycling) pattern and exaptational change representing human action

According to some embodiments of the present invention the alert module comprise the step of comparing in real time the accumulated data pattern to the templates patterns stored in the system, in a time adjustment period, when a change is discovered and the system is classified it as a human behavior change it updates the appropriate template stored in the memory.

According to some embodiments of the present invention the pattern analysis module is configured to:
identifying and determining, synchronization, correlation and synergy between different data types;
identifying and analyzing data patterns based on identified synchronization, correlation and synergy, based on personalized history data clustered data of peer users, environmental data/context data using learning algorithm.

According to some embodiments of the present invention the alert module further comprises creating learning AI models for each type of data (electricity, water flow, gas, communication, RSSI intensity, training model to identify change in user behavior based on each data type patterns.

According to some embodiments of the present invention the analysis modules further comprising the step of aggregating data from plurality of users, creating clusters of users based on user profile personal information including at least one of: geographical data, user behavior, medical data.

According to some embodiments of the present invention the analysis modules is implemented on cloud server.

According to some embodiments of the present invention the analysis modules is implemented on house hold End unit devices.

The present provides a method for monitoring and alert, implemented by plurality of modules comprising one or more processors, operatively coupled to non-transitory computer readable storage devices, comprising the steps of:
aggregating and/or synchronizing, filtering and calculating intensity changes and or creating data patterns template for each data type from data meters of electricity, waterflow gas data and all types of data communication flow
   identifying and determining, synchronization, correlation and synergy between different data types;
   identifying and Analyzing data patterns based on identified synchronization, correlation and synergy, based on personalized history data clustered data of peer users, environmental data/context data using learning algorithm;
determining alerts based on identified abnormality in data patterns.
According to some embodiments of the present invention further comprising the step of analyzing user movement based on RSSI measurements.
According to some embodiments of the present invention further comprising the step of identifying real time human activity and their association with the hours of the day by identifying Specific events that reflect human activities of opening or closing or changing setting of electrical gas or water appliance differentiating between repeating (cycling) pattern and exaptational change representing human action
According to some embodiments of the present invention the determining of alerts comprise comparing in real time the accumulated data pattern to the templates patterns stored in the system in a time adjustment period, when a change is discovered and the system is classified it as a human behavior change it updates the appropriate template stored in the memory
According to some embodiments of the present invention the determining of alerts comprises creating learning AI models for each type of data including electricity, water flow, gas, communication or RSSI intensity, training model to identify change in user behavior based on each data type patterns.

According to some embodiments of the present invention comprising the step of aggregating data from plurality of users, creating clusters of users based on user profile personal information comprising at least one of: geographical data, user behavior, medical data.

### BRIEF DESCRIPTION OF DRAWINGS

FIG. 1 is a block diagram illustrating monitoring and alert system according to some embodiments of the present invention.

FIG. 2 is a block diagram illustrating layout of RSSI (Received Signal Strength Indicator) received from the metering devices according to some embodiments of the present invention.

FIG. 3A is an illustration flow chart of electricity meter analyse module 30 according to some embodiments of the preset invention.

FIG. 3B is an illustration flow chart of water flow analysis module 32 according to some embodiments of the preset invention.

FIG. 3C is an illustration flow chart of gas meter analyse module 34 according to some embodiments of the preset invention.

FIG. 3D is an illustration flow chart of communication data analysis module 36 according to some embodiments of the preset invention.

FIG. 3E is an illustration flow chart of RSSI data analysis module 35 according to some embodiments of the preset invention.

FIG. 4 is an illustration flow chart of Pattern analysis all measured parameters module 10 according to some embodiments of the preset invention.

FIG. 5 is an illustration flow chart of Ai module 46, according to some embodiments of the preset invention.

FIG. 6 is an illustration flow alert module 44 according to some embodiments of the preset invention.

FIG. 7 is an illustration flow clustering module 48 according to some embodiments of the preset invention.

FIG. 8 is an illustration flow RSSI module 42 according to some embodiments of the preset invention.

FIG. 9 is an illustration flow of the monitoring analysis, and alerting process according to some embodiments of the preset invention.

FIG. 10 is a block diagram illustrating monitoring and alert system according to some embodiments of the present invention, AI and Machine learning is done on the server.

FIG. 11 is a block diagram illustrating monitoring and alert system according to some embodiments of the present invention, AI and Machine learning is done on one of the end unit.

FIG. 12 is a block diagram illustrating installation of the electricity meter installation according to some embodiments of the present invention;

FIG. 13 is a block diagram illustrating the electricity meter main units according to some embodiments of the present invention;

FIG. 14 is a block diagram illustrate the water meter main units according to some embodiments of the present invention;

FIG. 15 is a block diagram illustrating installation of the water meter installation according to some embodiments of the present invention;

FIG. 16 is a block diagram illustrate the gas meter main units according to some embodiments of the present invention;

FIG. 17 is a block diagram illustrating installation of the gas meter installation according to some embodiments of the present invention;

FIG. 18 is a block diagram illustrating configuration installation example of the monitoring alerting systems according to some embodiments of the present invention;

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

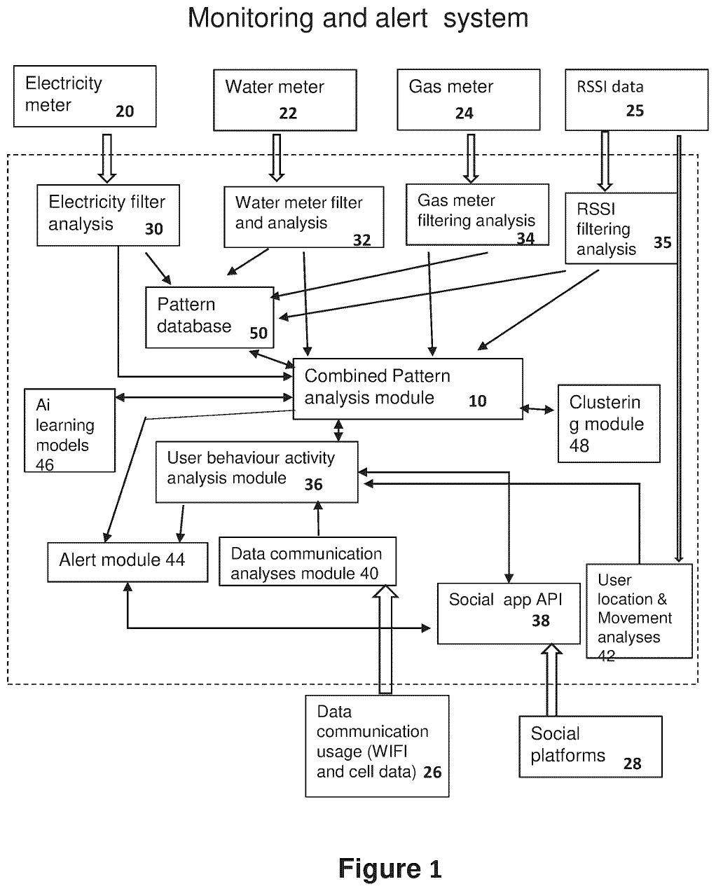

FIG. 1 is a block diagram illustrating monitoring and alert system according to some embodiments of the present invention.
The monitoring and alert system is comprised of:
Multiple metering device for measuring different data type: electricity meter 20, water flow meter 22, gas meter 24, communication data monitoring 26, RSSI data device monitoring 25
Multiple analysing and filtering modules for filtering and analysing different data types and creating data pattern templates representing each data type behavior over different time periods, the data patterns are saved in the data pattern database 50:
electricity filtering and analysing module 30, water filtering and analysing module 32, gas filtering and analysing module 34, communication data filtering and analysing module 40, RSSI data filtering and analysing module 35;
Combined Pattern analysis module 10 for integrating and analysing data of the different data types, this module analyses, correlation and synergy between data types, creating combined data patterns templates, analysing data using cluster data of peer user using clustering module 48 and applying AI leaning models 46;
User activity module 36 combines analysed data of data patterns and movements analysis using module 42;
Data flow measurement 26 measure the amount of data flow in real time and transfer the raw data flow to data communication analysis module 40

The alert model 44 determines alerts based on the pattern analysis and activating/behavior monitoring. The alert module further communicates with the social network API 38 for checking false alerts of use detected behavior using interaction on social media platforms 28.

There are two modes of operation first one: the information is transmitted to the server and all the analysis of the AI is done by the server. Alternatively, certain parts or all the AI processing is processed in the end units i.e. the water electricity and gas meter. One of the units is configured as the master unit, which manages the whole system, in this case the server is used as the supervisor only.

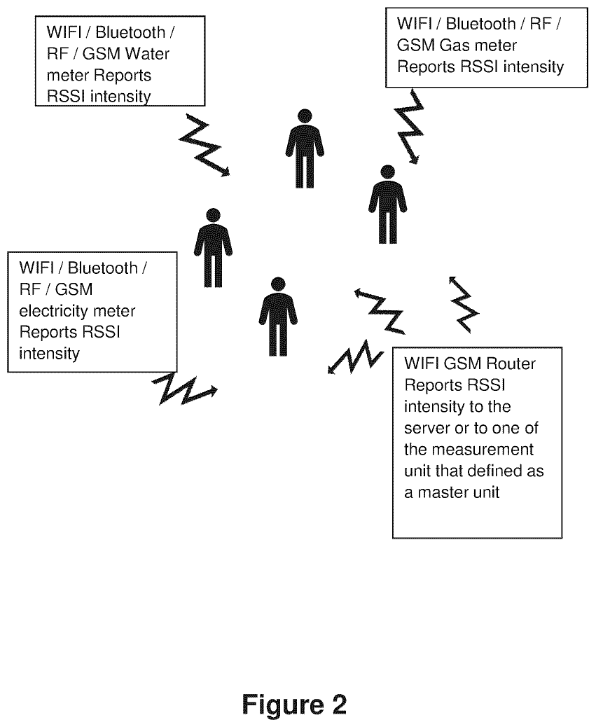

FIG. 2 is a block diagram illustrating the layout of RSSI metering devices according to some embodiments of the present invention.

The end units such as the meter device includes one or more of the following modems: RF/GSM/Bluetooth/WIFI. Each end unit reports the RSSI reception intensity. In normal mode the intensity is almost constant, when anybody passes between the transmitter and the receiver there is a change in the RSSII intensity decrease or increase in reception level.

The system learns the reception intensity level every X time and saves the data, A change in the envelope of the reception level indicates movement in the measured area. since the measurement devices are installed in different areas it is possible to reach full coverage of the measured area where the system installed.

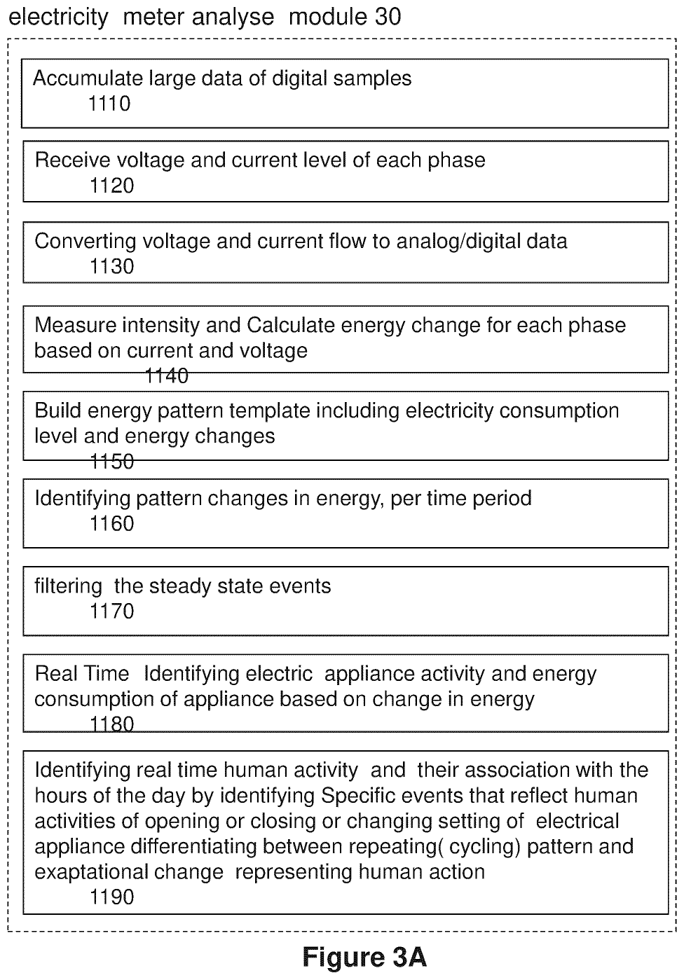

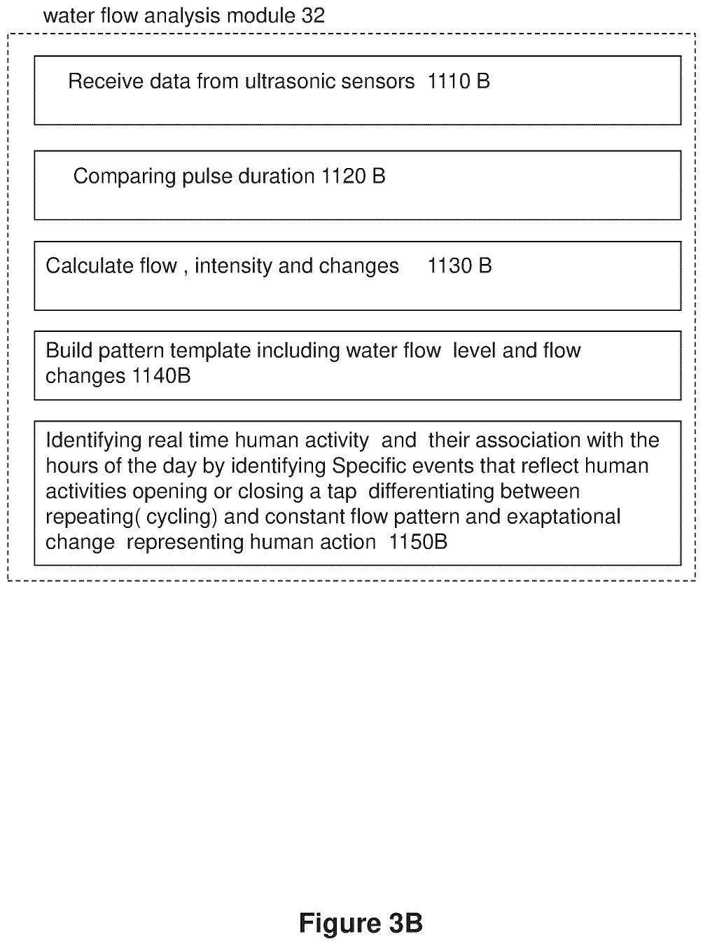

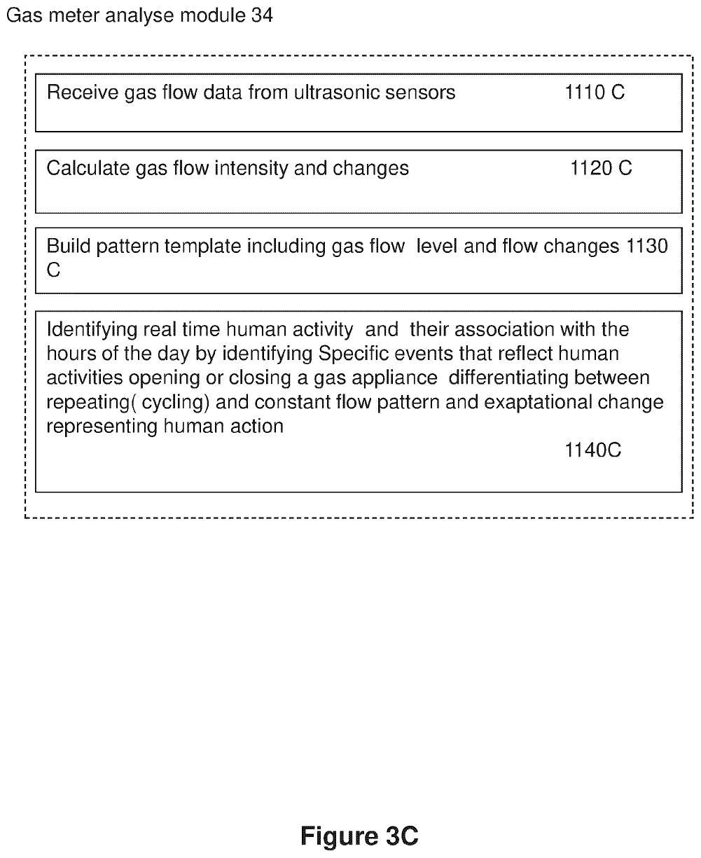

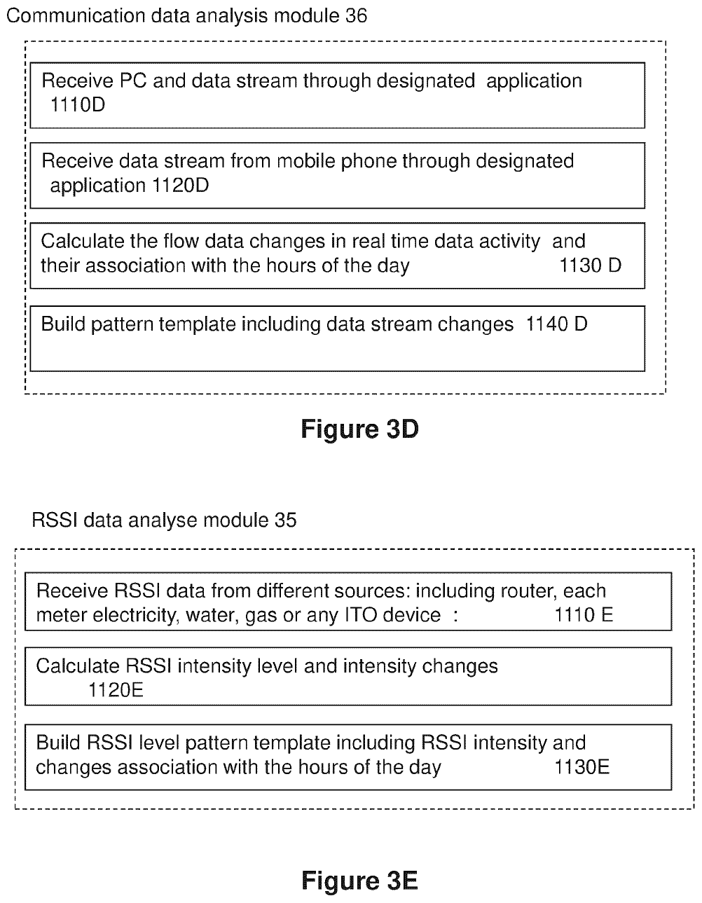

FIG. 3A is an illustration flow chart of electricity meter analyse module 30 according to some embodiments of the preset invention.

The electricity meter analyse module apply at least one of the following steps:
Accumulate large data of digital samples 1110;
Receive voltage and current level of each phase 1120;
Converting voltage and current flow to analog/digital data 1130;
Measure intensity and Calculate energy change for each phase based on current and voltage 1140;
Build energy pattern template including electricity consumption level and energy changes 1150;
Identifying pattern changes in energy, per time period 1160;
filtering the steady state events, 1170;
real time identifying electric appliance activity and energy consumption of appliance based on change in energy 1180;
Identifying in real time human activity and their association with the hours of the day by identifying Specific events that reflect human activities of opening or closing or changing setting of electrical appliance differentiating between repeating (cycling) pattern and exaptational change representing human action 1190;
FIG. 3B is an illustration flow chart of water flow analysis module 32 according to some embodiments of the preset invention.

The water meter analyse module apply at least one of the following steps:
Receive data from ultrasonic sensors 1110 B
Comparing pulse duration 1120 B
Calculate flow, intensity and changes 1130 B
Build pattern template including water flow level and changes 1140B
Identifying real time human activity and their association with the hours of the day by identifying Specific events that reflect human activities opening or closing a tap differentiating between repeating (cycling) and constant flow pattern and exaptational change representing human action 1150B;
FIG. 3C is an illustration flow chart of gas meter analyse module 34 according to some embodiments of the preset invention.

The gas meter analyse module apply at least one of the following steps:
Receive gas flow data from ultrasonic sensors 1110 C;
Calculate gas flow intensity and changes 1120 C;
Build pattern template including gas flow level and changes 1130 C;
Identifying real time human activity and their association with the hours of the day by identifying Specific events that reflect human activities opening or closing a gas appliance differentiating between repeating (cycling) and constant flow pattern and exaptational change representing human action 1140C;

FIG. 3D is an illustration flow chart of communication data analysis module 36 according to some embodiments of the preset invention.

The data communication analyse module apply at least one of the following steps:

Receive PC data stream through designated application 1110D

Receive data stream from mobile phone through designated application 1120D;

Calculate the flow data changes in real time data activity and their association with the hours of the day 1130 D;

Build pattern template including data stream changes 1140 D

FIG. 3E is an illustration flow chart of RSSI data analysis module 35 according to some embodiments of the preset invention.

The RSSI data analyse module apply at least one of the following steps:

Receive RSSI data from different sources: including router, each meter electricity, water, gas or any ITO device: 1110 E

Calculate intensity level and intensity changes 1120E;

Build RSSI level pattern template including RSSI intensity and changes association with the hours of the day 1130E

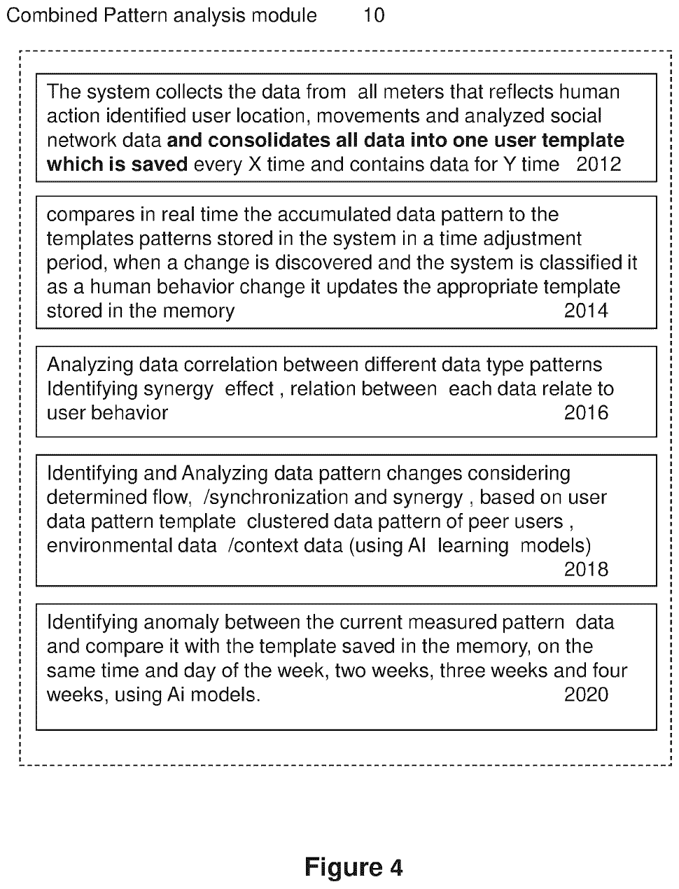

FIG. 4 is an illustration flow chart of Pattern analysis all measured parameters module 10 according to some embodiments of the preset invention.

The combined Pattern analysis module apply at least one of the following steps:
The system collects the data from all meters that reflects human action identified user location, movements and analyzed social network data and consolidates all data into one user template which is saved every X time and contains data for Y time 2012;
compares in real time the accumulated data pattern to the templates patterns stored in the system in a time adjustment period, when a change is discovered and the system is classified it as a human behavior change it updates the appropriate template stored in the memory 2014;

Analyzing data correlation between different data type patterns

Identifying, correlation and synergy effect, relation between each data relate to user behavior 2016;

Identifying and Analyzing data pattern changes considering determined flow/synchronization, correlation and synergy, based on user data pattern template clustered data pattern of peer users, environmental data/context data (using AI learning models); 2108

Identifying anomaly between the current measured pattern data and compare it with the template saved in the memory, on the same time and day of the week, two weeks and three weeks, using Ai models; 2020

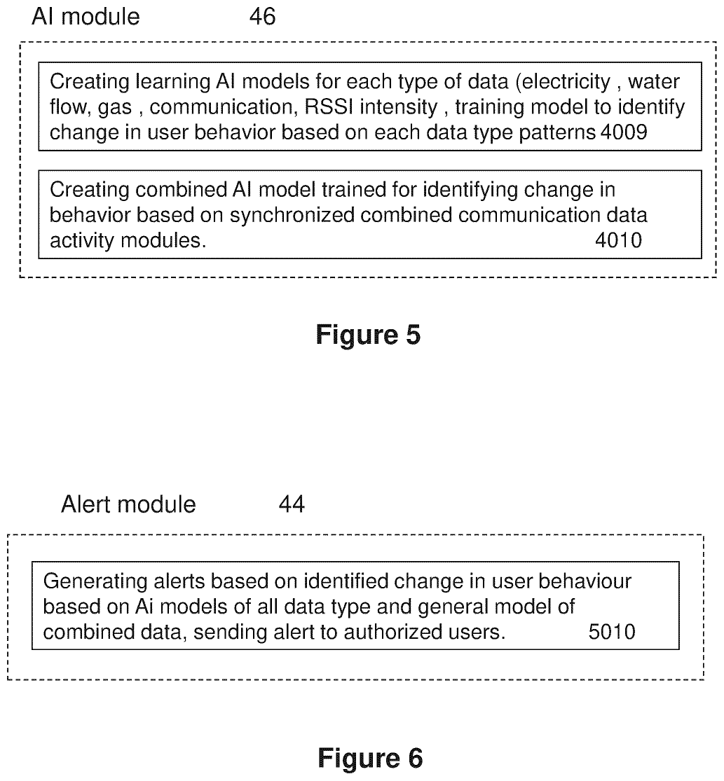

FIG. 5 is an illustration flow chart of Ai module 46, according to some embodiments of the preset invention.

Creating learning AI models for each type of data (electricity, water flow, gas, communication, RSSI intensity, training model to identify change in user behavior based on each data type patterns 4009.

Creating combined AI model trained for identifying change in behavior based on synchronized combined communication data activity modules. 4010

FIG. 6 is an illustration flow alert module according to some embodiments of the preset invention.

Generating alerts based on identified change in user behaviour based on Ai models of all data type and general model of combined data, sending alert to authorized users 5010

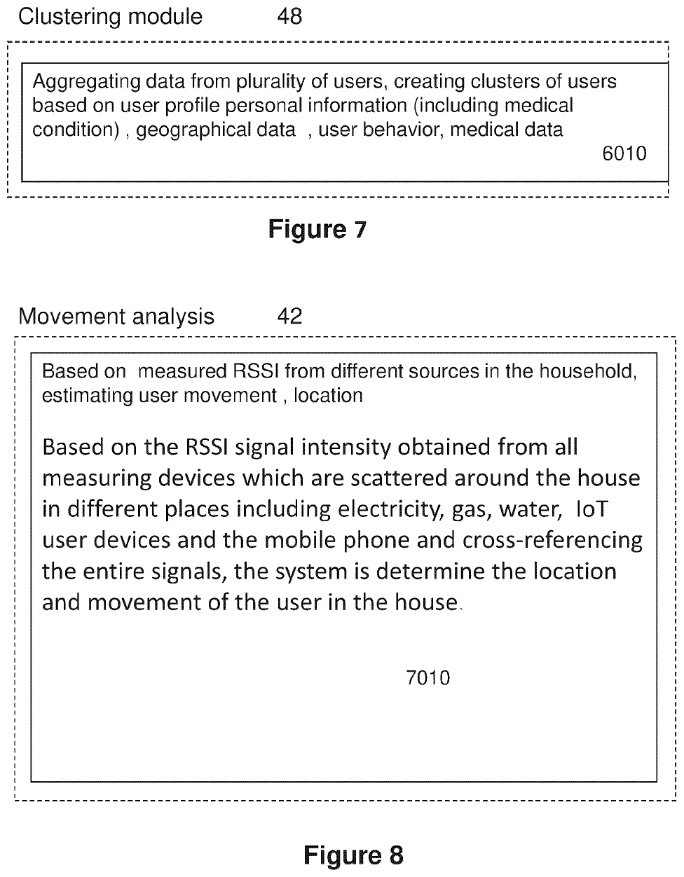

FIG. 7 is an illustration flow clustering module 48 according to some embodiments of the preset invention.

Aggregating data from plurality of users, creating clusters of users based on user profile personal information (including medical condition), geographical data, user behavior, medical data 6010;

FIG. 8 is an illustration movement analysis module 42 according to some embodiments of the preset invention.

Based on the RSSI signal intensity obtained from all measuring devices which are scattered around the house in different places including electricity, gas, water, IoT user devices and the mobile phone and cross-referencing the entire signals, the system is determine the location and movement of the user in the house.

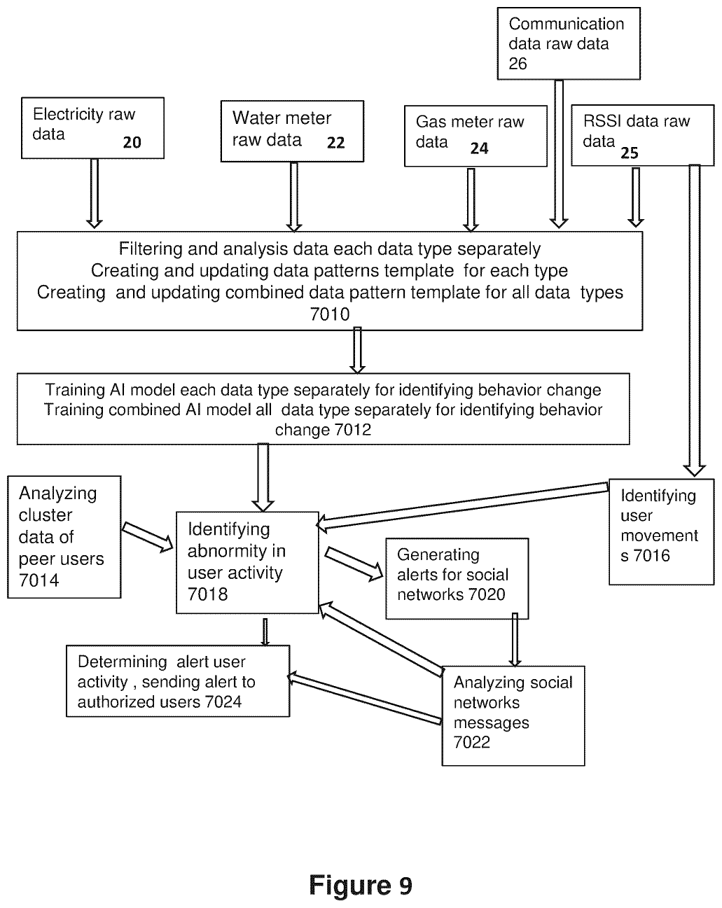

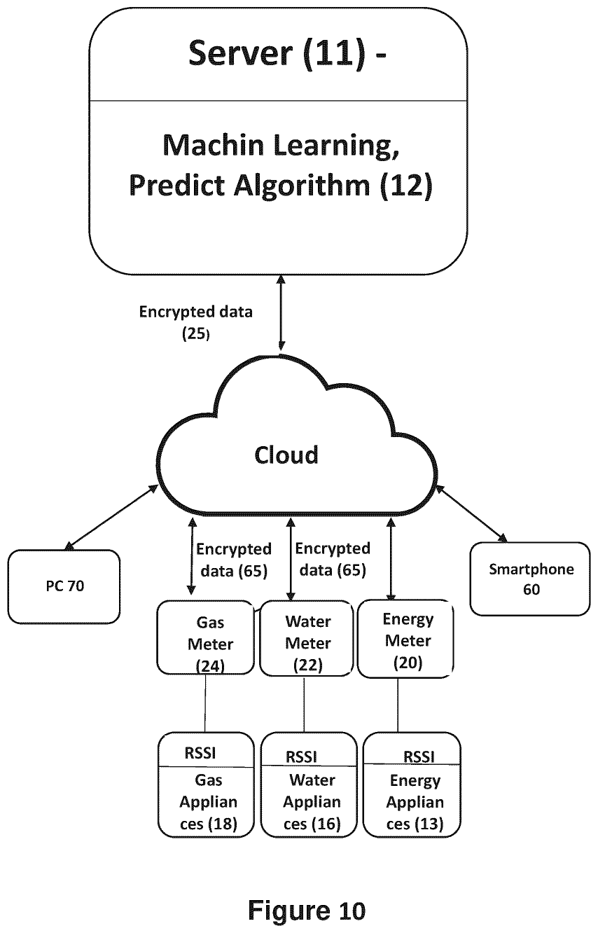

FIG. 9 is an illustration flow of the, AI analysis, monitoring and alerting process according to some embodiments of the preset invention.
The AI, monitoring and alert system process received data form the different meters Electricity raw data (20), Water meter raw data (22), Gas meter raw data
(24), RSSI data raw data (25), Communication data raw data (26)

and apply filtering (7010) and analysis data each data type separately for creating and updating data patterns template for each type. based on aggregated data for all different meters is created and updated combined data pattern template for all data types.

Based on AI Training model (7012 for each data type separately, the combined data model and Identifying user movements (7016) are identified behavior changes taking into account Analyzing cluster data of peer users (7014).

In cased of identified abnormity in user behavior change (7018), the change is tested in order determine is severity by generating alerts for social networks (7020), analyzing social networks messages (7022) and determining alert of user activity to be sent to authorized users (7024)

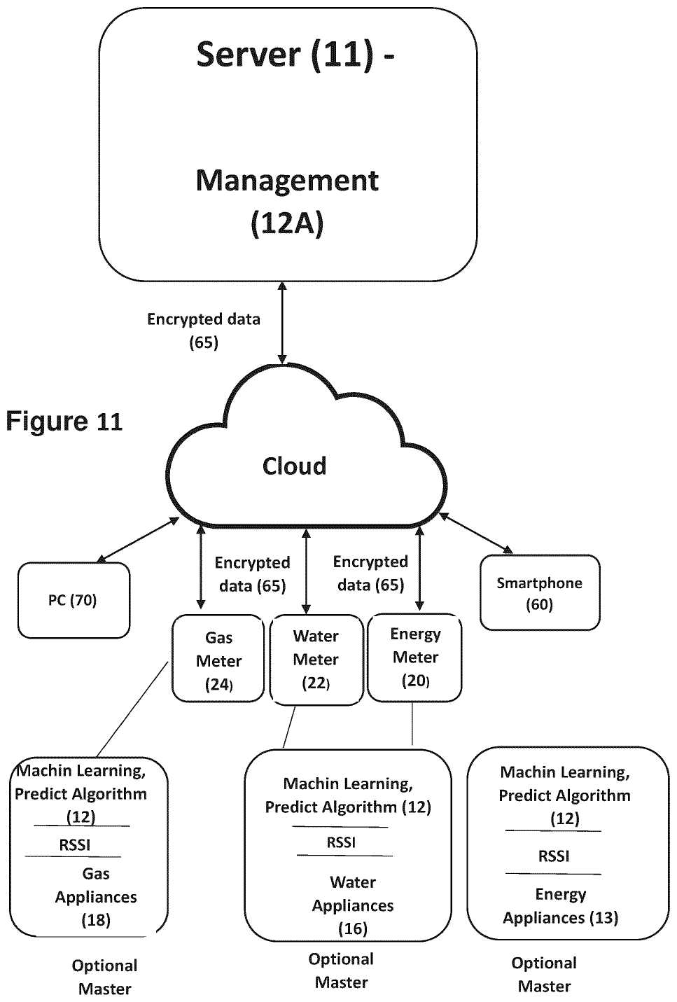

FIG. 11 is a block diagram illustrating the AI, monitoring and alert system according to some embodiments of the present invention.

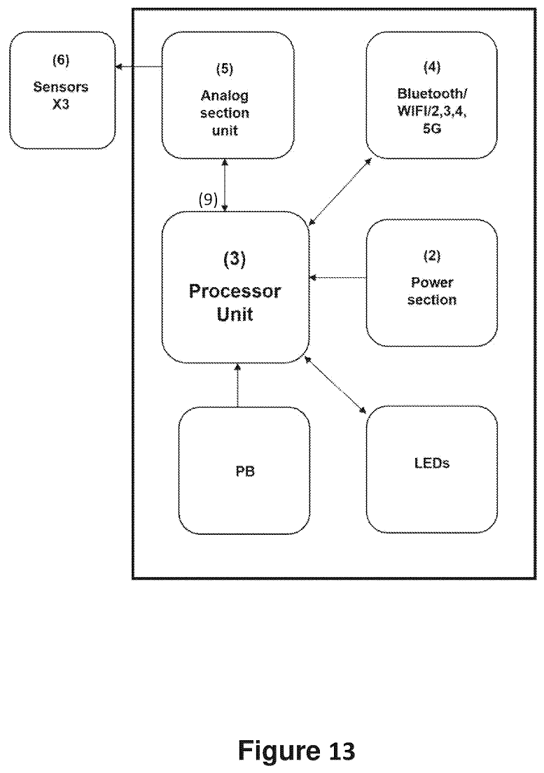

The Server (11)—receive all the measurement data from different meter devices including the electrical energy meter device 20, the water meter device 22, the gas meter 24 data communication and RSSI data from each meters at different IoT devices (the server control all meters via the Wi-Fi, Bluetooth, 2,3,4,5G (GSM) interface (4), (22) installed at the meter devices see FIG. 13, 14,16). The server (11) which receives all the information from the end units (???), activates a machine learning and prediction algorithm of the user's consumption behavior and updates the anomality table on the server and send alerts to the app on the smartphone (60) an/or the PC (70) through the cloud, the server (11) also sends SMS or WhatsApp messages if necessary to pre-defined phone numbers in the system.

The Smartphone (60) and/or PC (70) application receives/transmits the encrypted data (65) and control signals from/to the server and view the relevant screen include notification and alert status.

The application runs on a smartphone (60) or/and PC (70), communicate with the server (11) and receive the entire relevant information about the status of the user at a specific place where the system was installed and view the alerts regarding the activity of the users at home. The Data is encrypted, hence only the authorized person can see it.

The app displays on the Smartphone (60) and/or the PC (70) several levels of alerts, red color indicates a high alert level and will be accompanied by sending SMS or WhatsApp to a pre-defined phone numbers in the system. This alert is only sent after it has been observed that the user is currently at home is not behaving as expected, in an unusual way i.e., no activities have been observed based on the information collected and calculated by the AI algorithm. For example: in the morning in case there is no water flow it may indicate the user has not performed his regular activities such as brushing teeth or making coffee, which should include water consumption and energy for heating the water (Gas or electric), and also activating a number of electrical appliances, water or gas during the day.

The user has the option to set in alerts level on the app and define when it should be a red code (with or without SMS).

The dashboard on the Smartphone (60) or PC (70) application will show different color for each level of alert red will indicate urgent alert, green color will present “Good” status, orange, yellow, light green will indicate in-between alerts.

The present invention enables a prediction of a changes in the user habits at his home by identifying change is the data pattern for identifying an anomaly in his normal behavior.

The system enables remote monitoring in the event of inactivity or partial activity at the user home without infringing his privacy.

The server (11) receives measurements which indicates the operation of electrical appliances (13) and/or water appliances (16) and/or gas appliances (18) consumption, it processes the information received from all the devices (? (?) learns the behavior of the user consumption and compare it with the previous usage collected and saved in the server (11) it can predict the expected behavior of the user, and alert of him if there is not match with the expected behavior. The monitoring of the appliance's usage (13), (26)(18) is done continuously 24/7, received and saved in the server.

Based on this information, the system displays the status of the user's activity on the smartphone (60) and/or PC (70).

The application dashboard displays the messages: everything is fine, i.e., the user is behaving as expected, continuing with irregular behavior and ending with problematic behavior.

The Smartphone (60)/PC (70) dashboard shows green color when user is behaving as expected and red color when serious problem is detected in the user behavior, and additional several numbers of intermediate colors for a different level of activity.

Optionally the system may send an SMS or WhatsApp message to predefined phone numbers.

The data processing and the ability to identify exceptional situations, including machine learning, can be performed on the server (11) and/or in the end units (24), (22), (20).

FIG. 11 is a block diagram illustrating monitoring and alert system according to some embodiments of the present invention, wherein the AI and Machine learning is done on one of the end unit.

According to this embodiment, all analysis and calculation are processed by the end device 24, 22,20 each device comprising machine leaning and prediction algorithms. The server only coordinates data and control transfer between all devices.

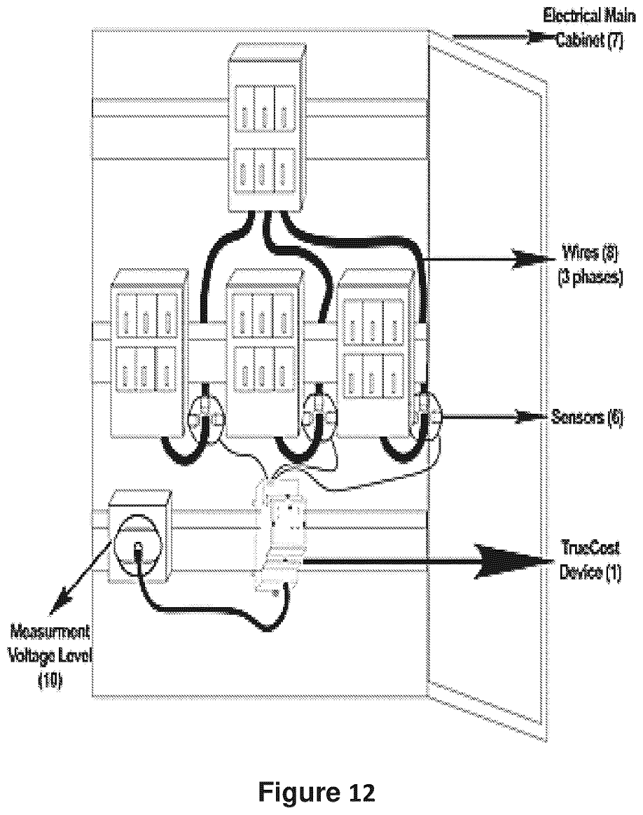

FIG. 12 is a block diagram illustrating installation of the electricity meter according to some embodiments of the present invention.

This figure, exemplify implementing the present invention is electricity meter in the electrical main cabinet 7: including: sensors 6 measurement current level, electricity meter (True Cost) device (1), measurement voltage level (10).

The electricity meter (TrueCost) device (1) has wireless communication.

The electricity meter (TrueCost) device (1) measures real-time electrical energy consumption on each phase, which can be displayed on PC (70) or Smartphone (60). Each electricity meter (TrueCost) measuring probe can be easily (Quick connect) connected to each phase without disconnecting the wires from the electrical system or shutting down power.

Installation is done within a few minutes, and after very short registration, the electricity meter (TrueCost) is ready and begins sending electrical energy consumption data directly to your PC (70) or Smartphone (60). Data is encrypted (25) so only the authorized user can view this data.

The intelligent Energy System analyzer that connects directly to your main electrical system (DIN standard) in the home main cabinet (7).

FIG. 13 is a block diagram illustrating the electricity meter according to some embodiments of the present invention.

The electricity meter device consists of the following units: Power section (2) Processor unit (3) Wireless interface Wi-Fi, Bluetooth, 2,3,4,5G (4), Analog section unit (5) that received the quick connect sensors (6) signal. The sensor (6) which is connected to the main supply wire (8) in the main cabinet (7) convert the inductance current that flow through the wire (8) the output of the sensor (6) is connected to the analog section unit (5) and converts the measured signal to the appropriate level of the processor unit (3) analog input (9).

The processor unit (3) samples the analog signal at a fast rate and cyclically, accumulate a large number of digital samples per second, in order to reach the maximum accuracy of the measured signal curve.

The processor unit (3) receives the voltage level (10) of each phase (can be one, two or three phases) from the analog section unit (5) the Voltage level is measured in the same manner as the current measurements (see above Section 1.a, b).

By having the real time current flow and the voltage level the processor unit (3) calculate the energy of each phase.

The calculated energy is transmitted to the server (11) via Wi-Fi, Bluetooth, 2,3,4,5G interface (4) in LoRa or/and proprietary protocol, data is transmitted every one second (the time interval of sending data from the device unit (1) to the cloud can be change from 100 mSec up to once a day)

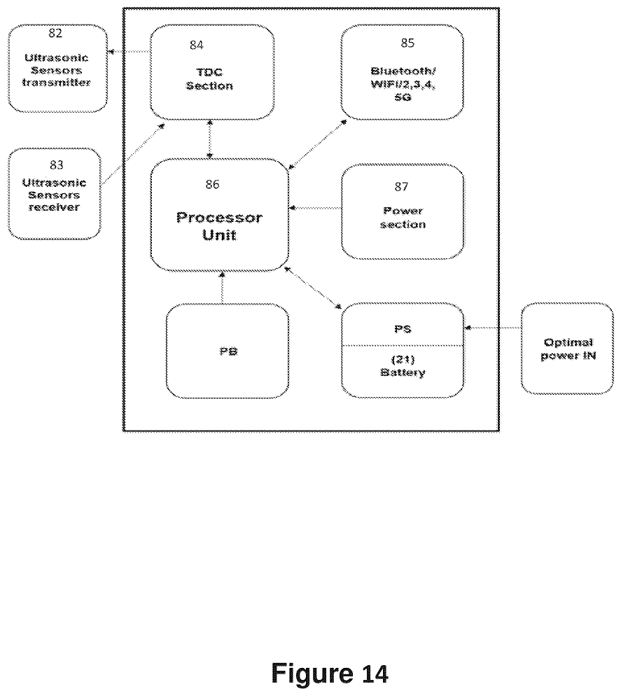

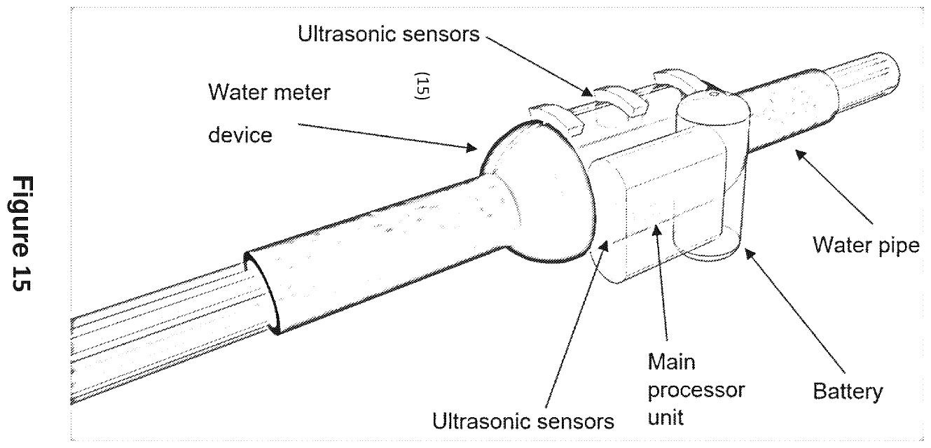

FIG. 14 is a block diagram illustrate the water meter according to some embodiments of the present invention;

The water meter device (22) is connected to the main water pipe (15) at the user home. The system consists of the following units: Power section (87) Processor unit (86) TDC (Time-to-Digital-Converters) section unit (84) two ultrasonic sensors transmitter (82) and Receiver (83).

Since the product is powered from battery (21) and in order to save power, sleep mode is the default state of the device. In sleep mode only the TDC unit (84) and the two ultrasonic sensors are in power on (waken up mode).

Receiver (82) and Transmitter (83) are connected to the TDC section (84) the TDC convert the water flow by comparing the pulse duration received from the ultrasonic sensors, on each pre define time (1 sec

and can be change according to the application needs) time it measured the water flow and only

when it detects water flow it wakeup the main processing unit (86), data is sampled on each pre define time, the processor unit (85) calculate the Water low intensity and transmit the data to the server via Wi-Fi, Bluetooth, 2,3,4,5G interface (22) in LoRa or/And proprietary protocol.

The server (11) receives the water flow measurement data and run an algorithm (12) (optional the algorithm can run also on the device processor unit (86) without involving the server) that characterizes the use of the water at the user's home, the information is continuously updated so after a few days it can be well characterized how the end customer uses the water at home, with each passing day the system is updated and give more accurate prediction of the user habits.

FIG. 15 is a block diagram illustrating installation of the water meter according to some embodiments of the present invention.

Water meter Device (22)—is wireless water meter and installed on the main water pipe (15) the installation is done within a few minutes with a quick connect prob, the device measures the water flow in the user home and transmits the encrypted data (65) to the server (11), the server (11) is able to control the unit (14) and change the device measurement mode. The device (14) is in sleep mode by default, and wakeup when it detects a water flow.

Smartphone (60)/PC (70) application receives/transmits the encrypted data (65) and control signals from/to the server and view the relevant screen include notification and alert status.

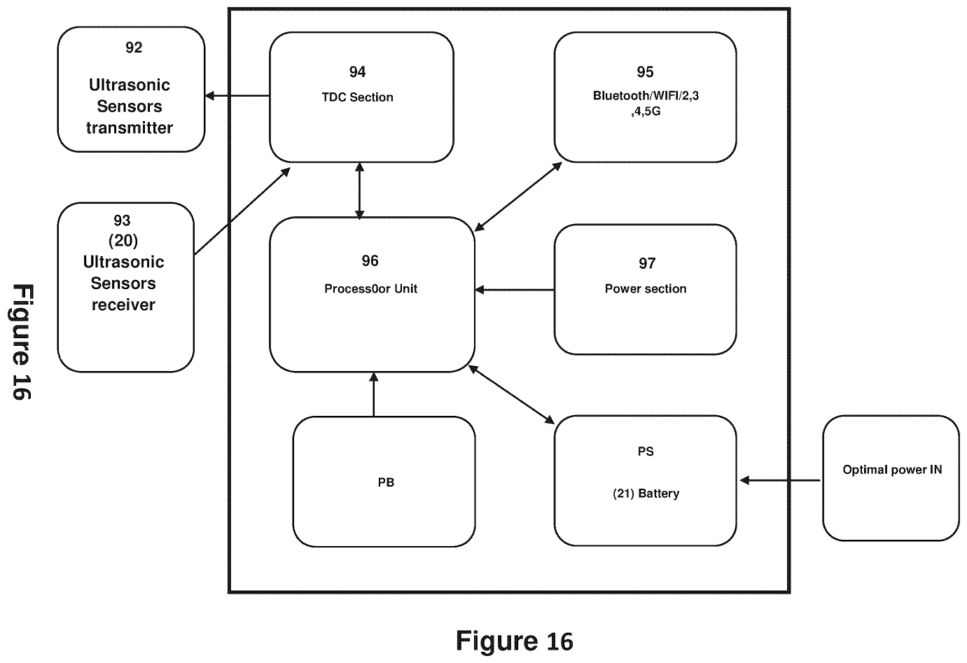

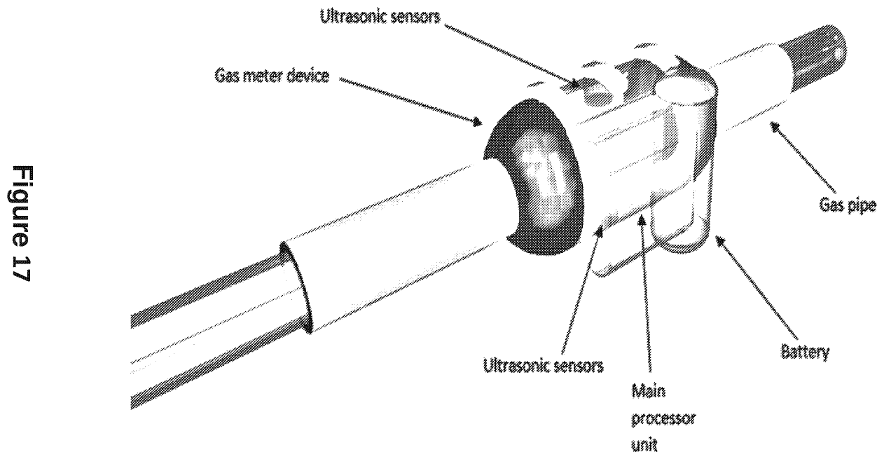

FIG. 16 is a block diagram illustrate the gas meter according to some embodiments of the present invention.

The gas meter device (24) is connected to the main water pipe (17) at the user home. The system consists of the following units: Power section (87) Processor unit (86) TDC (Time-to-Digital-Converters) section unit (84) two ultrasonic sensors transmitter (82) and Receiver (83).

Since the product is powered from battery (21) and in order to save power, sleep mode is the default state of the device. In sleep mode only the TDC unit (84) and the two ultrasonic sensors are wakening up.

Receiver (82) and Transmitter (83) are connected to the TDC section (84) the TDC convert the gas flow by comparing the pulse duration received from the ultrasonic sensors, on each pre define time (1 sec and can be change according to the application needs) time it measured the water flow and only when it detects gas flow its wakeup the main processing unit (86), data is sampled on each TBD sec, the processor unit (85) calculate the gas low intensity and transmit the data to the server, via Wi-Fi, Bluetooth, 2,3,4,5G interface (22) in LoRa or/And proprietary protocol.

The server (11) receives the gas flow measurement data and run an algorithm (12) (optional the algorithm can run also on the device processor unit (86) without involving the server) that characterizes the use of the gas at the user's home, the information is continuously updated so after a few days it can be well characterized how the end customer uses the water at home, with each passing day the system is updated and more give more accurate prediction.

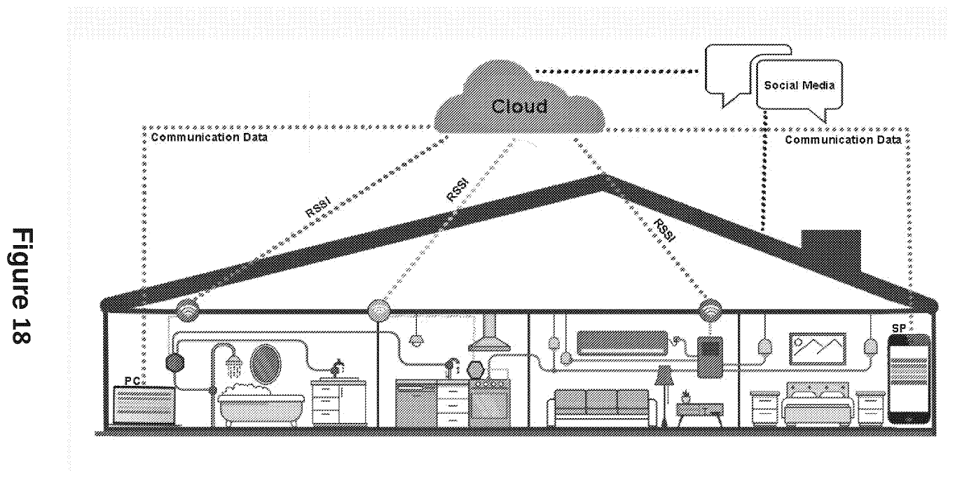

FIG. 17 is a block diagram illustrating installation of the gas meter according to some embodiments of the present invention.
gas meter Device (24)—is wireless water meter and installed on the main gas pipe (15) the installation is done within a few minutes with a quick connect prob, the device measures the gas flow in the user home and transmits the encrypted data (65) to the server (11), the server (11) is able to control the unit (14) and change the device measurement mode. The device is in sleep mode by default, and wakeup when it detects a water flow.

FIG. 18 is a block diagram illustrating configuration installation example of the RSSI communication network according to some embodiments of the present invention.

The RSSI data is collected from various data source od sensors and meter device located at various places in house such as: the bathroom, kitchen, living room or bedroom.

The system of the present invention may include, according to certain embodiments of the invention, machine readable memory containing or otherwise storing a program of instructions which, when executed by the machine, implements some or all of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may wherever suitably operate on signals representative of physical objects or substances.

Unless specifically stated otherwise, as apparent from the following discussions, it is appreciated that throughout the specification discussions, utilizing terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining” or the like, refer to the action and/or processes of a computer or computing system, or processor or similar electronic computing device, that manipulate and/or transform data represented as physical, such as electronic, quantities within the computing system's registers and/or memories, into other data similarly represented as physical quantities within the computing system's memories, registers or other such information storage, transmission or display devices. The term “computer” should be broadly construed to cover any kind of electronic device with data processing capabilities, including, by way of non-limiting example, personal computers, servers, computing system, communication devices, processors (e.g. digital signal processor (DSP), microcontrollers, field programmable gate array (FPGA), application specific integrated circuit (ASIC), etc.) and other electronic computing devices.

The present invention may be described, merely for clarity, in terms of terminology specific to particular programming languages, operating systems, browsers, system versions, individual products, and the like. It will be appreciated that this terminology is intended to convey general principles of operation clearly and briefly, by way of example, and is not intended to limit the scope of the invention to any particular programming language, operating system, browser, system version, or individual product.

It is appreciated that software components of the present invention including programs and data may, if desired, be implemented in ROM (read only memory) form including CD-ROMs, EPROMs and EEPROMs, or may be stored in any other suitable typically non-transitory computer-readable medium such as but not limited to disks of various kinds, cards of various kinds and RAMs. Components described herein as software may, alternatively, be implemented wholly or partly in hardware, if desired, using conventional techniques. Conversely, components described herein as hardware may, alternatively, be implemented wholly or partly in software, if desired, using conventional techniques.

Included in the scope of the present invention, inter alia, are electromagnetic signals carrying computer-readable instructions for performing any or all of the steps of any of the methods shown and described herein, in any suitable order; machine-readable instructions for performing any or all of the steps of any of the methods shown and described herein, in any suitable order; program storage devices readable by machine, tangibly embodying a program of instructions executable by the machine to perform any or all of the steps of any of the methods shown and described herein, in any suitable order; a computer program product comprising a computer useable medium having computer readable program code, such as executable code, having embodied therein, and/or including computer readable program code for performing, any or all of the steps of any of the methods shown and described herein, in any suitable order; any technical effects brought about by any or all of the steps of any of the methods shown and described herein, when performed in any suitable order; any suitable apparatus or device or combination of such, programmed to perform, alone or in combination, any or all of the steps of any of the methods shown and described herein, in any suitable order; electronic devices each including a processor and a cooperating input device and/or output device and operative to perform in software any steps shown and described herein; information storage devices or physical records, such as disks or hard drives, causing a computer or other device to be configured so as to carry out any or all of the steps of any of the methods shown and described herein, in any suitable order; a program pre-stored e.g. in memory or on an information network such as the Internet, before or after being downloaded, which embodies any or all of the steps of any of the methods shown and described herein, in any suitable order, and the method of uploading or downloading such, and a system including server/s and/or client/s for using such; and hardware which performs any or all of the steps of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any step described herein may be computer-implemented. The invention shown and described herein may include (a) using a computerized method to identify a solution to any of the problems or for any of the objectives described herein, the solution optionally include at least one of a decision, an action, a product, a service or any other information described herein that impacts, in a positive manner, a problem or objectives described herein; and (b) outputting the solution.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Features of the present invention which are described in the context of separate embodiments may also be provided in combination in a single embodiment.

For example, a system embodiment is intended to include a corresponding process embodiment. Also, each system embodiment is intended to include a server-centered “view” or client centered “view”, or “view” from any other node of the system, of the entire functionality of the system, computer-readable medium, apparatus, including only those functionalities performed at that server or client or node.

1. A system for monitoring and alert, implemented by plurality of modules comprising one or more processors, operatively coupled to non-transitory computer readable storage devices, comprising the steps of:
analysis module configured for aggregating and/or synchronizing, filtering and calculating intensity changes and or creating data patterns template for each data type from data meters of electricity, waterflow gas data and all types of data communication flow;
wherein the communication flow comprises receive data stream between different type of IoT device or mobile communication device and at least one router;
wherein the analysis and creation of data patterns comprises calculating said received flow stream data changes in real time data activity and their association with the hours of the day;
data pattern analyse module, configured to identify anomaly between the current measured pattern data and compare it with the template saved in the memory, on the same time and day of the week, two weeks or three weeks; and
alert module configured for determining alerts based on identifying anomaly in data patterns.

2. The system of claim 1, wherein the analysis module further comprising the step of analyzing user movement based on all types of wireless devices (modems) RSSI measurements wherein the RSSI measurements comprise the RSSI reception intensity between at least one IoT device and at least on RF/GSM/Bluetooth/WIFI device configured to detect a change in the RSSII intensity indicating of human body movements between at least one IoT device and RF/GSM/Bluetooth/WIFI device.

3. The system of claim 1, wherein the analysis module further comprising the step of identifying real time human activity and their association with the hours of the day by identifying specific events that reflect human activities of opening or closing or changing setting of electrical appliance, gas and water for differentiating between repeating (cycling) pattern and exaptational change representing human action

4. The system of claim 1, wherein the alert module comprises the step of comparing in real time the accumulated data pattern to the templates patterns stored in the system, in a time adjustment period, when a change is discovered and the system is classified it as a human behavior change it updates the appropriate template stored in the memory.

5. The system of claim 1, wherein the pattern analysis module is configured
identifying and determining, synchronization, correlation and synergy between different data types;
identifying and analyzing combined data patterns based on identified synchronization, correlation and synergy, based on personalized history data clustered data of peer users, environmental data/context data using learning algorithm.

6. The system of claim 1, wherein the alert module further comprises creating learning AI models for each type of data (electricity, water flow, gas, communication, RSSI intensity, training model) to identify change in user behavior based on each data type patterns.

7. The system of claim 1, wherein the analysis modules further comprising the step of aggregating data from plurality of users, creating clusters of users based on user profile personal information including at least one of: geographical data, user behavior, medical data.

8. The system of claim 1, wherein the analysis modules is implemented on cloud server.

9. The system of claim 1, wherein the analysis modules is implemented on house hold End unit devices.

10. A method for monitoring and alert, implemented by plurality of modules comprising one or more processors, operatively coupled to non-transitory computer readable storage devices, comprising the steps of:
aggregating and/or synchronizing, filtering and calculating intensity changes and or creating data patterns template for each data type from data meters of electricity, waterflow gas data and all types of data communication flow;
identifying and determining, synchronization, correlation and synergy between different data types;
identifying and analyzing data patterns based on identified synchronization, correlation and synergy, based on personalized history data clustered data of peer users, environmental data/context data using learning algorithm; and
determining alerts based on identified abnormality in data patterns.

11. The method of claim 10, further comprising the step of analyzing user movement based on RSSI measurements.

12. The method of claim 10, further comprising the step of identifying real time human activity and their association with the hours of the day by identifying Specific events that reflect human activities of opening or closing or changing setting of electrical gas or water appliance differentiating between repeating (cycling) pattern and exaptational change representing human action.

13. The method of claim 10, wherein the determining of alerts comprises comparing in real time the accumulated data pattern to the templates patterns stored in the system in a time adjustment period, when a change is discovered and the system is classified it as a human behavior change it updates the appropriate template stored in the memory

14. The method of claim 10, wherein the determining of alerts comprises creating learning AI models for each type of data including electricity, water flow, gas, communication or RSSI intensity, training model to identify change in user behavior based on each data type patterns.

15. The method of claim 10, further comprising the step of aggregating data from plurality of users, creating clusters of users based on user profile personal information comprising at least one of: geographical data, user behavior, medical data.
